(19) United States
(12) Patent Application Publication
(10) Pub. No.: US 2006/0213505 A1
(43) Pub. Date: Sep. 28, 2006

(54) Inhalation device

(75) Inventors: Darren Hodson, Shropshire (GB); Jorgen Rasmussen, Struer (DK)

Correspondence Address: NIXON & VANDERHYE, PC, 901 NORTH GLEBE ROAD, 11TH FLOOR, ARLINGTON, VA, 22203, US

(73) Assignee: AstraZeneca AB, Sodertalje (SE)

(21) Appl. No.: 11/346,559

(22) Filed: Feb. 3, 2006

Related U.S. Application Data
(63) Continuation-in-part of application No. 10/698,950, filed on Nov. 3, 2003.
Application No. 10/698,950 is a continuation of application No. 09/986,941, filed on Nov. 13, 2001, now abandoned.
Application No. 09/986,941 is a continuation of application No. 09/214,757, filed on Jan. 12, 1999, now abandoned.

(30) Foreign Application Priority Data
Nov. 14, 1997 (SE) 9704185-9

(51) Int. Cl. A61M 11/00

(52) U.S. Cl. 128/200.14

(57) ABSTRACT

An actuator for an inhaler for delivering medicament by inhalation, comprising: a main body comprising a tubular member for receiving a canister containing medicament and having a valve stem extending therefrom, a bottom surface at one end of the tubular member; a mouthpiece for guiding medicament to the mouth of a user; and a nozzle block located in the bottom region of the tubular member for receiving the valve stem of the canister and delivering medicament from the canister into the mouthpiece; wherein the bottom surface is formed as a foot which is configured such that, with a canister fitted therein, the actuator will stand unsupported with the tubular member extending generally vertically. There is also disclosed an inhaler comprising the above-described actuator and a canister containing medicament.

The present invention relates to an actuator for an inhaler for administering medicament by inhalation and to an inhaler including the same.

For some time, actuators have been known for delivering metered doses of medicament from aerosol canisters. Conventional actuators comprises a main body comprising a tubular member for receiving a canister containing medicament and having a valve stem extending therefrom, a bottom surface at one end of the tubular member; a mouthpiece for guiding medicament to the mouth of a user; and a nozzle block located in the bottom region of the tubular member for receiving the valve stem of the canister and delivering medicament from the canister into the mouthpiece. During inhalation, it is essential that the actuator be held in an upright position with the valve end of the canister pointed downward. This is to ensure that the valve openings, situated at the base of the valve are in contact with the medicament during use. Overall performance of the inhaler may be improved if it is orientated in the upright position as often as possible including the time between inhalations and storage. Moreover, storage in the upright position reinforces patient compliance with the desired orientation by which the inhaler must be used.

It is an aim of the present invention to provide an actuator with a unique construction that improves the performance of the inhaler.

Accordingly, the present invention provides an actuator for an inhaler for delivering medicament by inhalation, comprising: a main body comprising a tubular member for receiving a canister containing medicament and having a valve stem extending therefrom, a bottom surface at one end of the tubular member; a mouthpiece for guiding medicament to the mouth of a user; and a nozzle block located in the bottom region of the tubular member for receiving the valve stem of the canister and delivering medicament from the canister into the mouthpiece; wherein the bottom surface is formed as a foot which is configured such that, with a canister fitted therein, the actuator will stand unsupported with the tubular member extending generally vertically.

In one embodiment the bottom surface of the foot includes a recess for receiving a thumb or a finger of a user. Preferably, the recess is concave.

In another embodiment the bottom surface of the foot is flat.

According to one embodiment, the actuator comprises one or both of a breath actuation mechanism and a compliance monitor, in particular a dose counter. Preferably, the foot comprises one or both of the breath actuation mechanism and the compliance monitor.

According to one embodiment, the nozzle block includes a bore having an opening for receiving the valve stem of a canister and a spray orifice configured to direct a spray into the mouthpiece.

According to one embodiment, the actuator comprises a main body comprising the tubular member; and an outlet assembly, as a part formed separately of the main body, comprising the mouthpiece and the nozzle block.

According to one embodiment, the foot is formed as a part of the main body. According to another embodiment, the foot is formed as a part of the outlet assembly.

Preferably, the main body and the outlet assembly are configured so as to snap-fit together in that they are provided with complementary snap catch members.

According to one embodiment, at least a part of the outlet assembly is configured so as to deform and optionally break on withdrawal of the outlet assembly from the main body so as to prevent re-use of the inhaler.

The present invention also extends to an inhaler comprising the above-described actuator and a canister containing medicament.

Preferably, the inhaler is a pressurised metered dose inhaler.

A preferred embodiment of the present invention will now be described herein below by way of example only with reference to the accompanying drawings, in which:

FIG. 1 illustrates a perspective view of an inhaler in accordance with a preferred embodiment of the present invention;

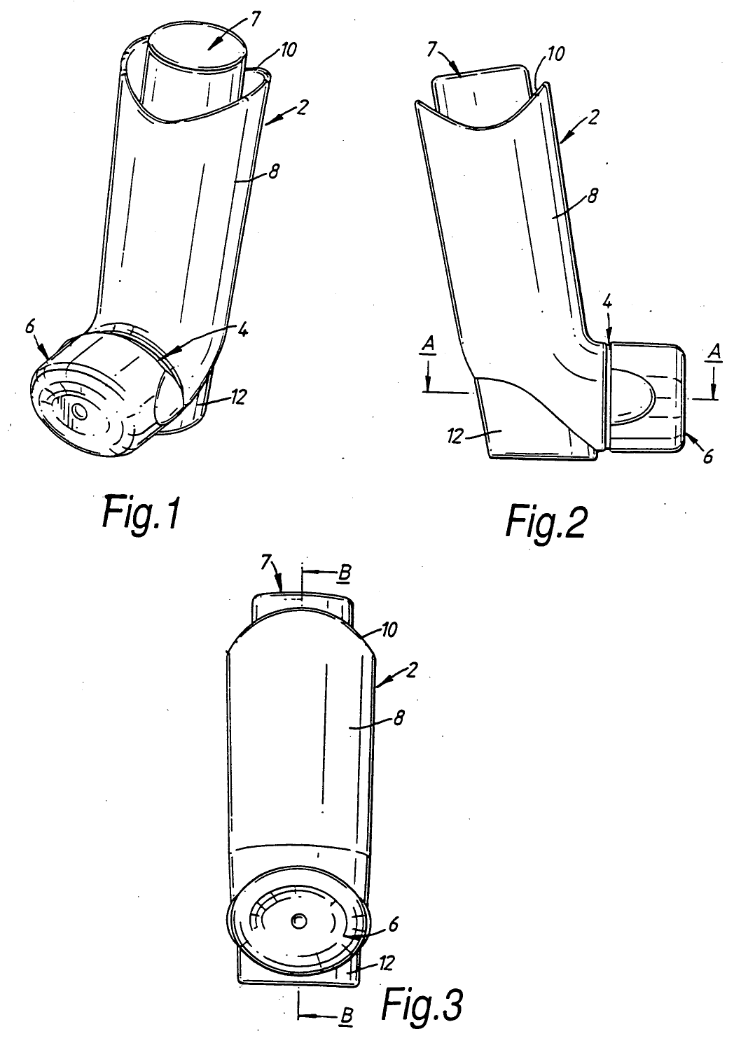

FIG. 2 illustrates a side view of the inhaler of FIG. 1;

FIG. 3 illustrates a front view of the inhaler of FIG. 1;

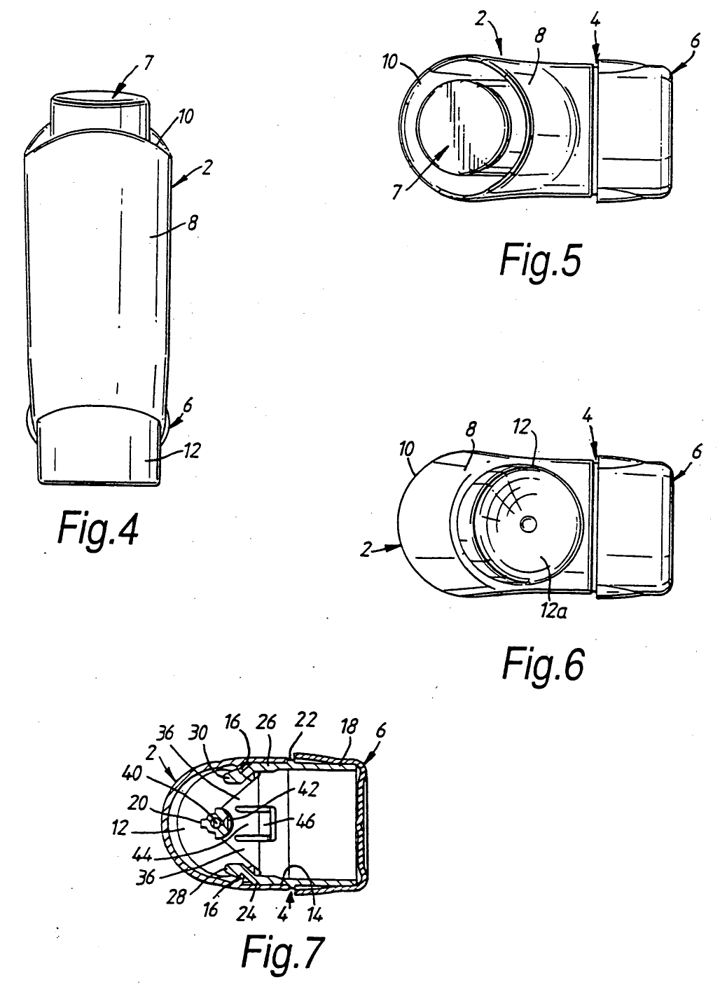

FIG. 4 illustrates a rear view of the inhaler of FIG. 1;

FIG. 5 illustrates a plan view of the inhaler of FIG. 1;

FIG. 6 illustrates an underneath plan view of the inhaler of FIG. 1;

FIG. 7 illustrates a horizontal sectional view (along section A-A) of the inhaler of FIG. 1;

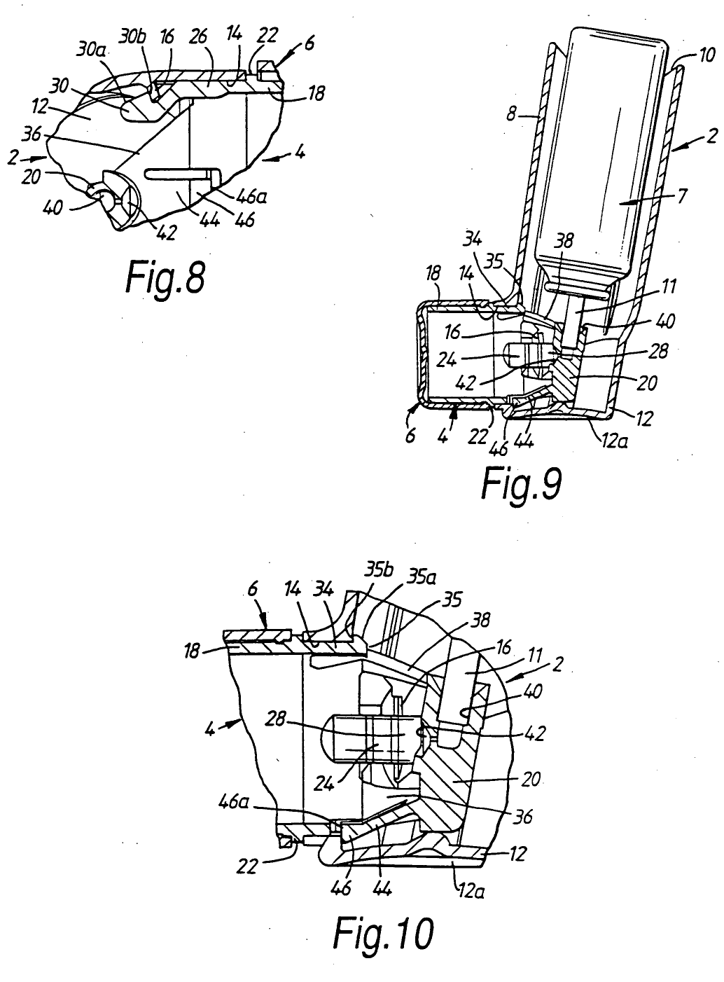

FIG. 8 illustrates in enlarged scale a fragmentary view of the section illustrated in FIG. 7;

FIG. 9 illustrates a vertical sectional view (along section B-B) of the inhaler of FIG. 1;

FIG. 10 illustrates in enlarged scale a fragmentary view of the section illustrated in FIG. 9;

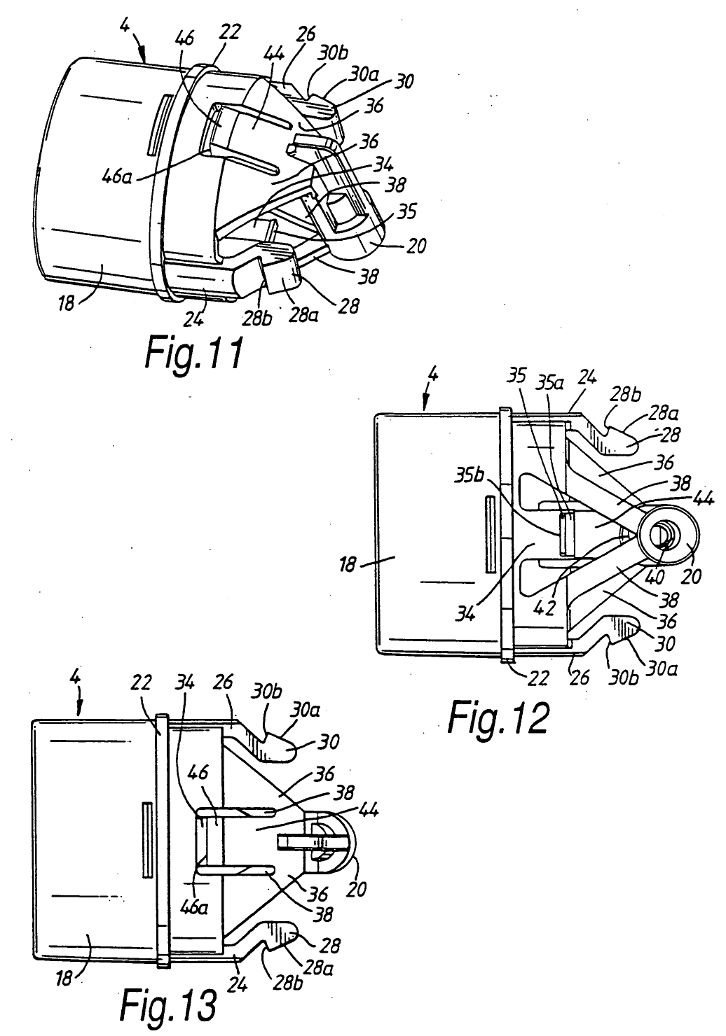

FIG. 11 illustrates a perspective view of the outlet assembly of the actuator of the inhaler of FIG. 1;

FIG. 12 illustrates a plan view of the outlet assembly of FIG. 11;

FIG. 13 illustrates an underneath plan view of the outlet assembly of FIG. 11;

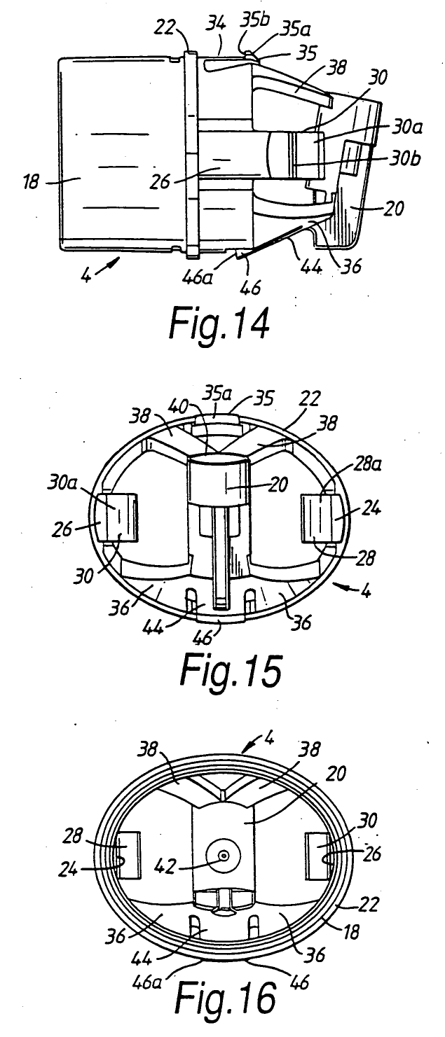

FIG. 14 illustrates a side view of the outlet assembly of FIG. 11;

FIG. 15 illustrates a rear view of the outlet assembly of FIG. 11; and

FIG. 16 illustrates a front view of the outlet assembly of FIG. 11.

The inhaler comprises an actuator, which comprises a main body 2, an outlet assembly 4 fitted to a lower part of the main body 2 and a cap 6, and an aerosol canister 7 containing medicament fitted therein.

The main body 2 comprises a tubular member 8 having an opening 10 at one, the upper end thereof into which a canister 7 having a valve stem 11 extending therefrom is in use fitted, and a foot 12 having a bottom surface which includes a recess 12a, in this embodiment concave in shape, for receiving typically a thumb of a user. In an alternative embodiment the foot 12 can be formed with a substantially flat bottom surface. The foot 12 serves to allow the actuator to stand unsupported on a flat surface such that, when the actuator is not in use, it can be stored in an upright position. This is particularly advantageous when a canister 7 is fitted therein, since such canisters 7 should, ideally, be stored with the valve stem 11 directed downwards. The other, lower, end of the tubular member 8 is closed and includes a lateral opening 14, in this embodiment ovoid in shape, into which the outlet assembly 4 is fitted.

The main body 2 further comprises a pair of opposing projections 16 which extend inwardly from the inner surface of the tubular member 8 adjacent the lateral opening 14. The projections 16 are disposed to the sides of the lateral opening 14 and are spaced rearwardly therefrom.

The outlet assembly 4 comprises a tubular section 18, a major part of which defines the mouthpiece which is in use gripped by the lips of a user, and a nozzle block 20 connected thereto.

The tubular section 18 includes a radial outwardly-directed peripheral flange 22. When the outlet assembly 4 is inserted fully into the main body 2, the flange 22 abuts the lateral opening 14 such that the major part of the tubular section 18 extends outwardly of the main body 2.

The outlet assembly 4 further comprises first and second arms 24, 26 which extend rearwardly form respective sides of the tubular section 18. Each of the first and second arms 24, 26 includes a catch member 28, 30 which is adapted to engage with a respective one of the projections 16 on the inner surface of the tubular member 8 when the outlet assembly 4 is inserted fully into the main body 2. The catch members 28, 30 on the first and second arms 24, 26 each include a first surface 28a, 30a which has a rearwardly directed component and acts as a guiding surface, and a second surface 28b, 30b which is substantially orthogonally directed to the longitudinal axis of the outlet assembly 4 and acts as a locking surface.

The outlet assembly 4 further comprises a third arm 34 which extends rearwardly from the top of the tubular section 18. The third arm 34 includes a catch member 35 in the form of an outwardly-directed projection, which, when the outlet assembly 4 is inserted fully into the main body 2, engages behind a part of the tubular member 8 defining the lateral opening 14. The catch member 35 on the third arm 34, as with the catch members 28, 30 on the first and second arms 24, 26, includes a first surface 35a which has a rearwardly directed component and acts as a guiding surface, and a second surface 35b which is substantially orthogonally directed to the longitudinal axis of the outlet assembly 4 and acts as a locking surface.

The nozzle block 20 is connected to the tubular section 18 by first and second pairs of connecting elements 36, 38. The first pair of connecting elements 36 extend between a lower part of the nozzle block 20 and a lower part of the tubular section 18. As will be described herein below, in this embodiment the lower connecting elements 36 are configured to break or be permanently deformed on withdrawal of the outlet assembly 4 from the main body 2. The second pair of connecting elements 38 extend between an upper part of the nozzle block 20 and an upper part of the tubular section 18. The nozzle block 20 includes a tubular bore 40 which extends along the longitudinal axis of the tubular member 8 when the outlet assembly 4 is inserted fully into the main body 2. The tubular bore 40 is open at one, the upper, end and includes a laterally-directed spray orifice 42 at the other, lower, end. The spray orifice 42 is configured to direct a spray into the tubular section 18. In this embodiment the tubular bore 40 is adapted to receive the valve stem 11 of a canister 7.

The outlet assembly 4 further comprises a fourth arm 44 which extends forwardly and downwardly from the nozzle block 20. The distal end of the fourth arm 44 includes a catch member 46 which, when the outlet assembly 4 is inserted fully into the main body 2, engages behind a part of the tubular member 8 defining the lateral opening 14. The catch member 46 on the fourth arm 44 includes a surface 46a which is substantially orthogonally directed to the longitudinal axis of the outlet assembly 4 and acts as a locking surface.

In manufacture, an outlet assembly 4 and a main body 2 are selected according to the requirements, based on colour, shape, etc., for the actuator. The outlet assembly 4 is then inserted into the lateral opening 14 in the main body 2 until the catch members 28, 30 on the first and second arms 24, 26 of the outlet assembly 4 engage with the respective projections 16 on the inner side surface of the tubular member 8 of the main body 2, and the catch members 34, 46 on the third and fourth arms 34, 44 of the outlet assembly 4 engage behind respective parts of the tubular member 8 defining the lateral opening 14. A canister 7 is then passed into the tubular member 8 of the main body 2 through the upper opening 10 such that the valve stem 11 of the canister 7 is located in the tubular bore 40 in the nozzle block 20. The inhaler is then ready for use.

By the provision of catch members the outlet assembly 4 is held in the main body 2 and the outlet assembly 4 cannot be non-destruct ably detached from the main body 2. As mentioned herein above, the outlet assembly 4 is configured to break or be permanently deformed if withdrawn from the main body 2 and thereby render the outlet assembly 4 and hence the actuator unusable. In this embodiment this is achieved by configuring the lower connecting elements 36 connecting the tubular section 18 and the nozzle block 20 of the outlet assembly 4 to break or be permanently deformed on withdrawal of the outlet assembly 4 from the main body 2.

Finally, it will be understood by a person skilled in the art that the present invention is not limited to the described embodiment but can be modified in many different ways within the scope of the appended claims.

1. An actuator for an inhaler for delivering medicament by inhalation, comprising: a main body comprising a tubular member for receiving a canister containing medicament and having a valve stem extending therefrom, a bottom surface at one end of the tubular member; a mouthpiece for guiding medicament to the mouth of a user; and a nozzle block located in the bottom region of the tubular member for receiving the valve stem of the canister and delivering medicament from the canister into the mouthpiece; wherein the bottom surface is formed as a foot which is configured such that, with a canister fitted therein, the actuator will stand unsupported with the tubular member extending generally vertically.

2. The actuator according to claim 1, wherein the bottom surface of the foot includes a recess for receiving a thumb or a finger of a user.

3. The actuator according to claim 2, wherein the recess is concave.

4. The actuator according to claim 2, wherein the bottom surface of the foot is flat.

5. The actuator according to claim 1, further comprising one or both of a breath actuation mechanism and a compliance monitor, in particular a dose counter.

6. The actuator according to claim 5, wherein the foot comprises one or both of the breath actuation mechanism and the compliance monitor.

7. The actuator according to claim 1, wherein the nozzle block includes a bore having an opening for receiving the valve stem of a canister and a spray orifice configured to direct a spray into the mouthpiece.

8. The actuator according to claim 1, wherein the actuator comprises a main body comprising the tubular member; and an outlet assembly, as a part formed separately of the main body, comprising the mouthpiece and the nozzle block.

9. The actuator according to claim 8, wherein the foot is formed as a part of the main body.

10. The actuator according to claim 8, wherein the foot is formed as a part of the outlet assembly.

11. The actuator according to claim 8, wherein the main body and the outlet assembly are configured so as to snap-fit together in that they are provided with complementary snap catch members.

12. The actuator according to claim 8, wherein at least a part of the outlet assembly is configured so as to deform and optionally break on withdrawal of the outlet assembly from the main body so as to prevent re-use of the inhaler.

13. An inhaler comprising the actuator according to claim 1 and a canister containing medicament.

14. The inhaler according to claim 13, wherein the inhaler is a pressurised metered dose inhaler.
